(19) United States
(12) Patent Application Publication
(10) Pub. No.: US 2007/0238698 A1
(43) Pub. Date: Oct. 11, 2007

(54) Method for manufacturing drinking water having chitosan

(75) Inventor: Hsien-Chih Lin, Taichung (TW)

Correspondence Address: HDSL, 4331 STEVENS BATTLE LANE, FAIRFAX, VA, 22033, US

(73) Assignees: Hsien-Chih Lin; Pure Earth Biotechnology Co., Ltd.

(21) Appl. No.: 11/399,325

(22) Filed: Apr. 7, 2006

(51) Int. Cl. A61K 31/722; A61K 31/7008

(52) U.S. Cl. 514/55; 514/62

(57) ABSTRACT

A method to make chitin dissolve into and stored in the acid water to obtain drinking water having chitosan includes putting water into chitin. By mixing or an adequate vibration, the drinking water having granular chitosan is produced. Further, the granular chitosan in the water has excellent preservative and stable properties.

### BACKGROUND OF THE INVENTION

### FIELD OF THE INVENTION

The present invention relates to a drinking water having granular chitosan and the method for manufacturing the same. More particularly, it relates to a method of forming drinking water having water-soluble chitosan with granular particles from chitin.

### BACKGROUND OF THE INVENTION

The drinking water is known valid for people to drink, its resources are: 1. tap water: sanitary water supplied by waterworks and water pipes to people through fillet. 2. water on earth: the water exists in streams, lakes, dams, ponds or other locations. 3. water under the ground: water exists in a water storage layer under the ground. They can be the drinking water shown by the form of package, such as: packaged mineral water, package distill water, packaged pure water or other drinking water package and sealed by the package which can not be recovered after being opened. Because the drinking water are drunk directly into human's body, therefore, the environmental or Health office only pay attention at the substance inside the drinking water is harmful to people or not. With respect to the people's drinking feeling to drinking water, the effect to activate the cell of human being . . . etc. are ignored.

It is also know to public that, chitin and chitosan are biological polymers made form natural substances. It has a well compatibility with biological cells. Moreover, it does not contain any poison, not only, can be dissolved by biological substances, but also has biological activities. Therefore, it is widely applicable to functional healthy food, medical appliances, processed food, cosmetics, textile, environment, agriculture, and chemistry etc.

However, fill now, there is no drinking water having chitosan for providing people with chitosan, so as to replenish the lost body tissue, increase people's immunity from inflection, and activate the cell of people.

Chitin is found in the exoskeletons of crustaceans, insect cuticles, organs of the mollusks, and fungal cell walls. The difference between chitin and chitosan is the degree of the deacetylation. Chitin is highly acetylated glucosamine. General speaking, chitin itself is not soluble in the water. Therefore, it requires a soluble medium for forming the water-soluble chitosan. In other words, the distinction between chitin and chitosan is based on the solubility in the dilute acid solution (for example, 2% acetic acid). That is in the mixture of chitin and chitosan under acetic acid, the soluble part is chitosan, and the insoluble part is chitin. FIG. 1 shows the structures of chitin and chitosan. In general, chitin is extracted from crustacean shell and it contains 15% of amine and 85% of acetyl group. When the deacetylation degree is over 70%, it can transform into the water-soluble chitosan which can be dissolved in the dilute acid solution. In other words, chitin having 70% and/or more of amine is called chitosan.

The water-soluble chitosan is commonly produced by the following process. First, the organic acid is added into the water-swollen chitin for dissolution. Therefore, chitin in a swollen state can be dissolved in the acid solution. However, the above process only adds chitin in the acid solution for forming a gel from the surface of chitin, while it is not completely dissolved into the water. Also, it requires other complicated process to complete, such as alkali thermal hardening method, and enzyme method.

According to the above prior art, even the chitin structure is adjusted, it is not easy for chitin to keep a liquid state during transmission. Furthermore, when chitin is preserved in a liquid state for a period, it is easy to cause deterioration. For example, the color becomes brown, and the liquid becomes turbid. Further, when the concentration of the liquid chitin is higher, it can cause a solid crystal form.

Consequently, it is not suitable to be need as drinking water, and it is then a loss to people.

### SUMMARY OF THE INVENTION

The main object of the present invention is to provide a drinking water having chitosan and the method for manufacturing the drinking water. By a simple manufacture method as mentioned in the present invention, chitosan can be dissolved into and stored in the acid water to obtain drinking water having chitosan, and the granular particles of chitosan is produced to have excellent preservative and stable properties.

Additional features and advantages of the invention will be set forth in the detailed description which follows, and in part will be readily apparent to those skilled in the art from the description or recognized by practicing the invention as described in the written description and claims hereof, as well as the appended drawings.

### BRIEF DESCRIPTION OF THE DRAWINGS

FIG. 1 shows the structures of chitin and chitosan;

FIG. 2 shows a structure formula of the polymer water-soluble chitosan;

FIG. 3 is a flow chart showing one example for manufacturing chitosan in the drinking water of the present invention;

FIG. 4 is a flow chart showing another example for manufacturing chitosan in the drinking water of the present invention;

FIG. 5 shows the structure of one granular particle in water-soluble chitosan in the drinking water of the present invention; and

FIG. 6 shows the structure of another granular particle in water-soluble chitosan in the drinking water of the present invention.

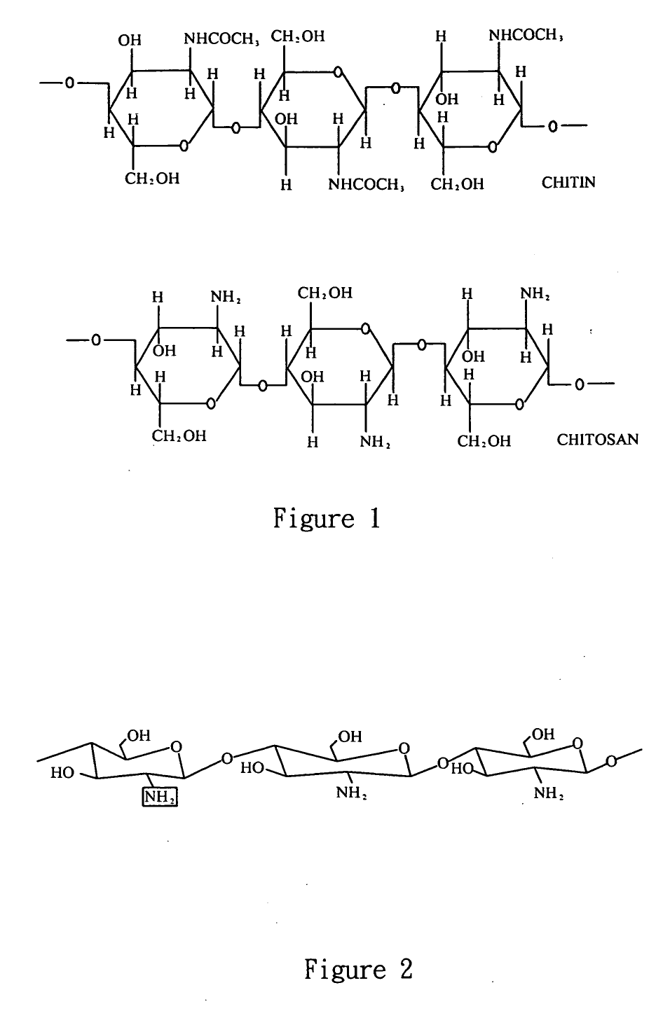

### PREFERRED EMBODIMENTS OF THE PRESENT INVENTION

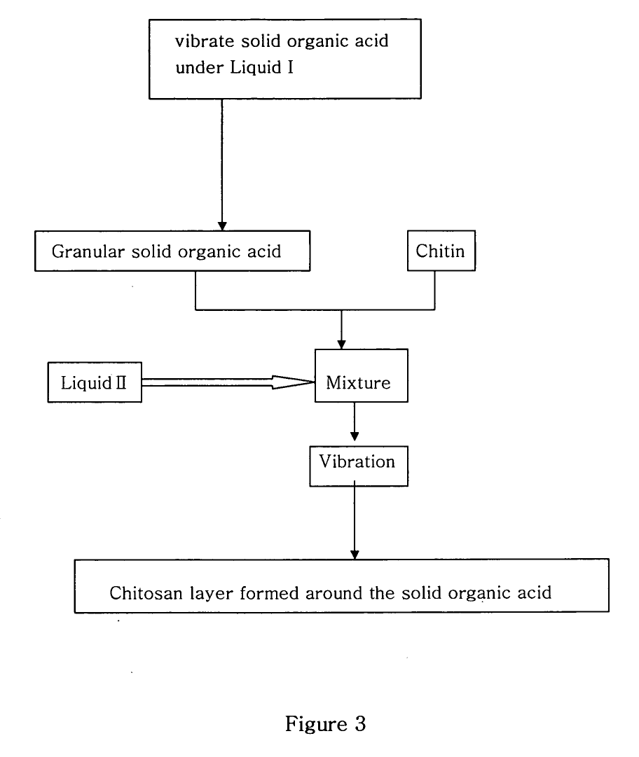

Please refer to FIG. 3. FIG. 3 is the method for manufacturing drinking water having chitosan of granular particles. It comprises: (1) the process of vibration the solid organic acid for forming grains under the existence of liquid I; and (2) the process of vibration the mixture of chitin and the granular solid organic acid under the existence of liquid II.

The chitin mentioned above is insoluble in water, however, the drinking water having chitosan of granular particles obtaining by the present invention can be dissolved in the water.

Chitosan generally can be dissolved in the organic and inorganic acid with acid or dilute acid, such as, acetic acid, formic acid, lactic acid, citric acid, pyruvic acid, hydrochloric acid, sulfuric acid, and phosphoric acid etc., wherein formic acid is the best dissolvent which can dissolve over 50% of chitosan.

FIG. 2 shows a structure formula of the polymer water-soluble chitosan. In the excellent preservative and stable properties, the granular water-soluble chitosan is preserved in a solid state by adhering around the organic acid in drinking water. It has a better stable preservation than the one in a liquid state. In other words, the granular chitosan in the drinking water of the present invention does not cause any problem which is happened in chitin in a liquid state. For example, the color becomes brown, and the liquid becomes turbid and causes a solid crystal form. The drinking water of present invention then shall not change it qualities, it provides excellent feeling to people when he drinks the water.

The water-soluble chitosan of the present invention is easier to transmit or manufacture compared to the liquid chitin.

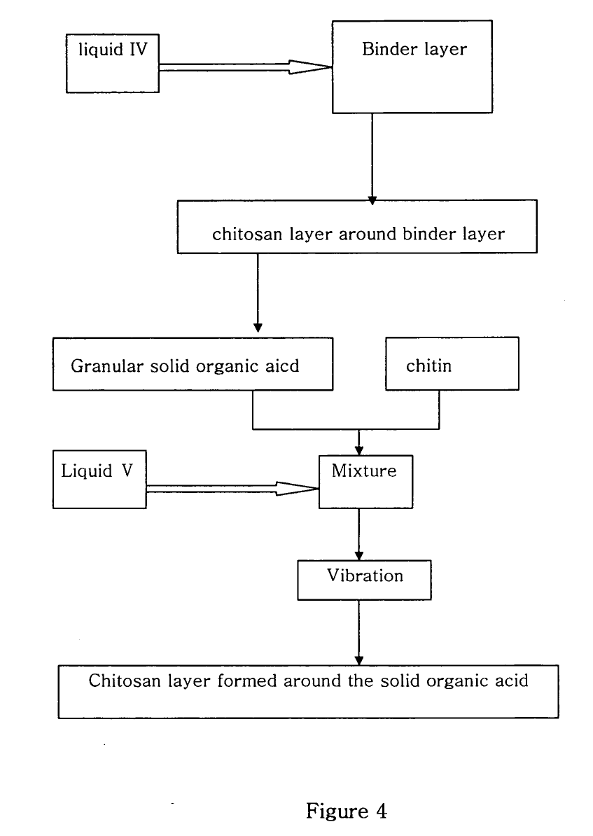

Please refer to FIG. 4. FIG. 4 is another preferred embodiment of the present invention showing a method for manufacturing water chitosan with granular particles of the drinking water of present invention. It comprises the steps of: (1) vibrating the solid organic acid forming grains under the existence of liquid III; (2) vibrating the mixture of a binder and above mentioned granular solid organic acid under the existence of liquid IV; and (3) vibrating the mixture of chitosan and above mentioned granular solid organic acid under the existence of liquid V

The water-soluble chitosan with granular particles (hereinafter referred to as “2^{nd} water-soluble chitosan”) obtained by the above preferred embodiment includes a binder layer. Since this water-soluble chitosan with granular particles chitosan includes a binder layer, the 2^{nd} water-soluble chitosan has a higher solubility while compared to the 1^{st} water-soluble chitosan.

The solid organic acid (molecular weight of the organic acid is 2000˜100,000) used for manufacturing the drinking water of the present invention is formed as crystal or powder for the best usage. Further, there is no specialized limitation for the type of the organic acid. For example, malic acid, citric acid, succinic acid, Malonic acid, maleic acid, and fumaric acid selected from polycarboxylic acid, and ascorbic acid all can be the organic acid for manufacturing the chitosan used in the drinking water of the present invention. And, the solid organic acid plays a core role in the water-soluble chitosan with granular particles. Further, the solid organic acid can be dissolved in the water as well as neutralize chitosan. It creates an environment for chitosan with an easier water-dissolution. In other words, the solid organic acid has a property for being neutralized or no PH state.

In order to make chitosan be dissolved completely, the best use quantity of the solid organic acid is higher than the quantity of glucosamine for neutralizing chitosan. Chitosan obtained by the method for manufacturing drinking water of present invention is made from chitin (poly-β-1,4-N-acetyl-D-glucosamine) and concentrated alkali solution after heating and then processing deacetylation. Chitosan is a polymer composition mainly formed by poly-β-1 and 4-N-acetyl-D-glucosamine. There is no specialized limitation on the molecular weight of chitosan. In the present invention, water-insoluble chitosan can be simply dissolved in the water. More particularly, the water-insoluble chitosan at 2000˜100,000 of molecular weight has an obvious effect. In deacetylation degree, there is no specialized limitation when the deacetylation degree is over 60%. More, the best application in the present invention is the deacetylation degree over 80%. In the size of chitosan, there is also no specialized limitation. The best size is over 40 meshes.

There are many kinds of binders applicable in the method for manufacturing drinking water of present invention. For example: oligosacchride, inositol, starch, dextrin, and dietary fiber ect. The above binders can be used individually or by mixture. General speaking, carbohydrate includes oligosacchride, inositol, and lactose. D-trehalose dehydrate, Isomaltase, D-raffinose, lactose are the products from oligosacchride. More, erythritol, and lactase are the products from inositol. Besides, fiber includes cellulose, hemi-cellulose, gumes, pectin, and lignin. For example, wheat flour, bran, cabbage, yong peas, broccoli, and peppers belongs cellulose of fiber.

The binder layer for manufacturing the drinking water of present invention is between the core of the organic acid and the chitosan layer. It has the following advantages:
1. When the water-soluble chitosan having granular particles is put into the water, chitosan will be swollen by the wet water. Later, the binder and the organic acid are dissolved in the water accordingly. Therefore, the water-soluble chitosan with the binder has a higher solubility.
2. The use quantity of the binder basically is various with the size of the grain. If the weight of the organic acid is 100, the use quantity of the binder is at 50˜100.

### EXAMPLE 1

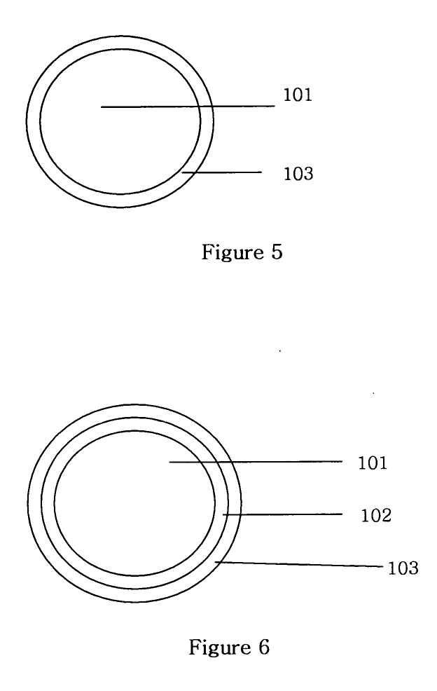

Please refer to FIG. 5; it is the first preferred embodiment of the structure of one granular particle of water-soluable chitosan in the drinking water of present invention. The first preferred embodiment of a method for manufacturing water-soluble chitosan with granular particles. It comprises the steps of: under existence of liquid I, the solid organic acid being vibrated for forming grains.

After forming the mixture of the granular solid organic acid and chitin, liquid II is added. In the meanwhile, the mixture is vibrated until the chitosan layer 103 is formed around the solid organic acid 101.

### EXAMPLE 2

Please refer to FIG. 6. It is the second preferred embodiment of the structure of one granular particle of water-soluable chitosan in the drinking water of present invention. The second preferred embodiment includes the water-soluble chitosan (hereinafter referred to as “2^{nd} water-soluble chitosan”) of granular particles.

The second preferred embodiment includes the granular water-soluble chitosan having the binder layer 102 around the solid organic acid 101. Further, the chitosan layer 103 is formed around the binder layer 102.

The method for manufacturing the 2^{nd} water-soluble chitosan comprises the steps of: 1. under the structure of liquid II, vibrating the mixture of chitin and the granular solid organic acid formed in the first manufacture method;
2. Adding liquid IV into the mixture of the granular solid organic acid and the binder formed from Example 1. In the meanwhile, the mixture is vibrated until the binder layer is formed around the solid organic acid;
3. Later, adding liquid V into the mixture of the solid organic acid with the formed binder layer and chitosan. In the meanwhile, the mixture is vibrated for forming the chitosan layer around the binder layer. Therefore, the “2^{nd} water-soluble chitosan of granular particle” is obtained.

The vibration method as mentioned in the above is commonly used. For example, a rotary granulator can be used as an adequate vibration method while manufacturing a large amount of granular water-soluble chitosan.

Liquid I used for manufacturing the water-soluble chitosan having granular particled of examples can be water or alcohol. The water or alcohol can be used individually or by mixture. In the type of alcohol, the lower boiling point is best for this type. For example, ethyl alcohol is the best liquid to use among methyl alcohol, ethyl alcohol, and propyl alcohol. When mixing the water and alcohol, the best mixture ration (ethyl alcohol:water) is 80:20. In addition, liquid I is the necessary for forming grains from the solid organic acid. When the weight of the solid organic acid is 100, the best use quantity of liquid I is at 10˜20.

General speaking, the preferred embodiment of liquid II is same as the one of liquid I . Therefore, when mixing the water and alcohol, the mixture ration of liquid II (ethyl alcohol:water) mostly is same as the ration of liquid I. Liquid II is the necessary substance for chitosan to adhere around the solid organic acid. When the weight of the solid organic acid is 100, the best use quantity of liquid II is at 10˜20.

The preferred embodiment of liquid III is same as liquid I. The preferred embodiment of liquid IV is same as liquid II. When mixing the water and alcohol, the best mixture ration (ethyl alcohol:water) is 80:20.

Liquid IV is the necessary substance for the binder to adhere around the solid organic acid. When the weight of the solid organic acid is 100, the best use quantity of liquid IV is at 10˜20.

The preferred embodiment of liquid V is same as the one of liquid II. When mixing the water with alcohol, the best mixture ration (ethyl alcohol:water) is 80:20. Liquid V is the necessary substance for chitosan to adhere around the solid organic acid. When the weight of the solid organic acid is at 100, the best use quantity of liquid V is at 10˜20.

### EXAMPLE 3

One of the preferred embodiments for obtaining chitosan in the method of manufacture drinking water of the present invention is that, 8 mg of the crystalline malic acid is added into the synthetic resin bag. Then, 1 liter of water by a spray method is added with vibration for 3˜5 minutes. Later, 10 mg of powder chitosan (molecular weight is 50,000˜60,000, and deacetylation degree is 80˜90%) is added. In addition, 3 liters of water by a spray method is added with vibration for 3˜5 minutes as forming the water-soluble chitosan having granular particles. Lastly, the grain is taken away from the bag. By using a dryer at 35° C. of constant temperature for 30 minutes, the granular size of the granular water-soluble chitosan after drying is about 20˜30 mesh.

Experimant I

In order to investigate the solubility of the granular water-soluble Chitosan, the present invention processes the following experiment I.

First, 1 mg of the water-soluble chitosan having granular particles obtained from the above manufacturing process is stirred by the stirrer for 3˜5 minutes after adding 100 liters of warm water (35° C.). After stirring, insoluble residuum is filtered. After residuum is dried, the residual ration is measured and obtained. The above manufacture is repeatedly processed for 5 times, and the residual ration from the measurement is between 0.5% and 0.8%.

### EXAMPLE 4

Another preferred embodiment for obtaining chitosan in the method of manufacturing drinking water of the present invention is that, 8 mg of the crystalline malic acid is added into the synthetic resin bag. Then, 1 liter of ethyl alcohol by a spray method is added with vibration for 3˜5 minutes. More, 5 mg of dextrin and 1 liter of ethyl alcohol by a spray method are added with vibration. Later, 10 mg of powder chitosan (molecular weight is 50,000˜60,000, and deacetylation degree is 80˜90%) is added. Furthermore, 6 liters of 80% of ethyl alcohol by a spray method is added with vibration for 3˜5 minutes as forming the granular water-soluble chitosan. Lastly, the grain is taken away from the bag. By using a dryer at 35° C. of constant temperature for 30 minutes, the granular size of the granular water-soluble chitosan after drying is about 20˜30 mesh.

Experiment II

In order to investigate the solubility of the granular water-soluble Chitosan as obtained from the above manufacture, the present invention again processes the following experiment II.

First, 1 mg of the granular water-soluble chitosan obtained from the above manufacture is stirred by the stirrer for 3˜5 minutes after adding 100 liters of warm water (35° C.). After stirring, insoluble residuum is filtered. After residuum is dried, the residual ration is measured and obtained. The above manufacture is repeatedly processed for 5 times, and the residual ration from the measurement is between 0.3% and 0.7%.

While the invention has been described with reference to preferred embodiments, it will be understood by those skilled in the art that various changes may be made and equivalents may be substituted for members thereof without departing from the scope of the invention. In addition, many modifications may be made to adapt a particular situation to the teachings of the invention without departing from the essential scope thereof. For example, it should be obvious that the slider guide may be formed as a monolithic piece or may be an assembly having two or more parts. Therefore it is intended that the invention not be limited to the particular embodiment disclosed as the best mode contemplated for carrying out this invention, but that the invention will include all embodiments falling within the scope of the appended claims.

### EXAMPLE 5

Add the chitosan obtained from the above examples into mineral water, distilled water or pure water, which can generate or cannot generate bubbles depending upon the existence of carbonic acid, then the waters becomes the drinking water having chitosan of present invention.

### EXAMPLE 6

The drinking water having chitosan obtained in Example 5 can be added with glucosamine, the ration is 1 L of drinking water to 0.01 g-10 g of glucosamine, preferably, 1 g-1.5 g of glucosamine. The addition of glucosamine shall make the drinking water more soft and smooth. Furthermore, glucosammine is the precursor for connecting cell and connective tissue. It is then has the functions of preventing skin aging, increasing the speed of injury healing, and is assistant for curing arthuristis.

### EXAMPLE 7

The drinking water having chitosan obtained in Example 5 can be added with chitosan oligosaccharide, the ratio is 1 L of drinking water to 0.01 g-5 g of chitosan oligosaccharide, preferably, 0.5 g-1 g of chitosan oligosaccharide. The addition of chitosan oligosaccharide shall reduced the hard feeling of drinking water to make the drinking water more soft and smooth. Furthermore, the addition of chitosan oligosaccharide shall increase people's immunity from inflection, activate the cell of people. By the experiments to animals, it also known that the addition of chitosan oligosaccharide shall reduced the inoculated tumors, increase the huge corrosion cell having ability of immunity and improve the enteric microflora of animals.

### EXAMPLE 8

The drinking water having chitosan obtained in Example 5 can be added with N-amino glucosamine, the ratio is 1 L of drinking water to 1 g-1.5 g of N-amino glucosamine, preferably, 0.5 g-1 g of N-amino glucosamine. The addition of N-amino glucosamine has the functions of depressing aging of people's skin, increase the speed of growing connection tissue and curing arthuristis.

### EXAMPLE 9

The drinking water having chitosan obtained in Example 5 can be added with chitin oligosaccharide, the ratio is 1 L of drinking water to 0.01 g-5 g of chitin oligosaccharide, preferably, 0.5 g-1 g of chitin oligosaccharide. The addition of chitin oligosaccharide not only can make the drink water become more delicate and delicious. And, inside the human body, the chitin oligosaccharide shall transfer to N-amino glucosamine, the drinking water then has the functions of depressing aging of people's skin, increase the speed of growing connection tissue and curing arthuristis.

Experiment III

A drinking water is obtained by using the process disclosed in Example 5 from 900 c.c. of mineral water having the following minerals, such as:
Cb 10.4/mg/L, Mg 2.8 mg/L, Na 4.0 mg/L, K 0.1 mg/L. And then, 1000 mg of glucosamine, 500 mg of chitosan oligosaccharide and 500 mg of Chitin oligosaccharide are respectively added into said drinking waters. The obtained drinking waters then are provided for 50 people to drink. In comparison with conventional mineral waters, the following evaluations are obtained:
   The drinking waters become much more delicate, much more smooth and much better fleeing to people.

The drinking waters become drinking waters without the feeling of hardness to people's mouth. And, people do not feel the existence of additives.

What is claimed is:

1. A method for manufacturing drinking water having chitosan, comprising the steps of:
(a) under the existence of a liquid I, vibrating solid organic acid for forming granular particles;
(b) under the existence of a liquid II, vibrating mixture of chitin and granular solid organic acid obtained in step (a), and
(c) adding the mixture obtained in step (b) into a drinking water which generates or does not generate bubbles.

2. The method as claimed in claim 1, wherein said water-soluble chitosan includes granular particles after processing step (b).

3. The method as claimed in claim 1, wherein said solid organic acid is in crystal or powder form.

4. The method as claimed in claim 1, wherein quantity of said solid organic acid is higher than quantity of glucosamine for neutralizing chitosan.

5. The method as claimed in claim 1, wherein said solid organic acid is polycarboxylic acid, or ascorbic acid.

6. The method as claimed in claim 1, wherein said liquid I or liquid II is formed by at least one selected from water or alcohol.

7. The method as claimed in claim 1, wherein said granular water-soluble chitosan is a chitosan layer formed around the solid organic acid.

8. The method as claimed in claim 1, wherein the drinking water is additively added with qlucosamine, or citosan, oligosaccharide, or N-aminoglucosamine, or chitin oligosaccharide.

9. The method as claimed in claim 8, wherein 0.01 g-10 g of glucosame, or citosan oligosaccharide, or N-aminoglucosamine, or chitin oligosaccharide is added to 1 liter of drinking water.

10. A method for manufacturing drinking water having chitosan, comprising the steps of:
(a) under the existence of a liquid III, vibrating solid organic acid for forming granular particles;
(b) under the existence of a liquid IV, vibrating mixture of binder and granular solid organic acid obtained in step (a);
(c) under the existence of a liquid V, vibrating mixture of chitosan and granular solid organic acid obtained in step (b); and
(d) adding substances obtained in step (c) into a drinking water which generates or does not generate bubbles.

11. The method as claimed in claim 10, wherein said water-soluble chitosan includes granular particles after step (c).

12. The method as claimed in claim 10, wherein said solid organic acid is in crystal or powder form.

13. The method as claimed in claim 10, wherein quantity of said solid organic acid is higher than quantity of glucosamine for neutralizing chitosan.

14. The method as claimed in claim 10, wherein said solid organic acid is polycarboxylic acid, or ascorbic acid.

15. The method as claimed in claim 10, wherein said liquid III, liquid IV or liquid V is formed by at least one selected from water or alcohol.

16. The method as claimed in claim 10, wherein said binder is formed by at least one selected from starch, carbohydrate, or oligosaccharide.

17. The method as claimed in claim 10, wherein said granular water-soluble chitosan is a chitosan layer formed around the solid organic acid.

18. The method as claimed in claim 10, wherein said granular water-soluble chitosan is a binder layer formed around the solid organic acid and chitosan layer formed around the binder layer.

19. The method as claimed in claim 10, wherein the drinking water is additively added with qlucosamine, or citosan, oligosaccharide, or N-aminoglucosamine, or chitin oligosaccharide.

20. The method as claimed in claim 19, wherein 0.01 g-10 g of glucosame, or citosan oligosaccharide, or N-aminoglucosamine, or chitin oligosaccharide is added to 1 liter of drinking water.
